(19) United States
(12) Patent Application Publication
(10) Pub. No.: US 2025/0248686 A1
(43) Pub. Date: Aug. 7, 2025

(54) MULTI-LUMEN DEVICE FOR ULTRASOUND IMAGING

(71) Applicant: Boston Scientific Scimed, Inc., Maple Grove, MN (US)

(72) Inventors: Austin Grant JOHNSON, Worcester, MA (US); Ryan McGRUDER, Overland Park, KS (US); Andrew James WHITNEY, Douglas, MA (US)

(73) Assignee: Boston Scientific Scimed, Inc., Maple Grove, MN (US)

(21) Appl. No.: 19/045,704

(22) Filed: Feb. 5, 2025

Related U.S. Application Data
(60) Provisional application No. 63/550,663, filed on Feb. 7, 2024.

(51) Int. Cl. A61B 8/00

(52) U.S. Cl. CPC A61B 8/445

(57) ABSTRACT

A medical device includes a central lumen extending from a proximal end to a distal end of the medical device that is configured to receive a probe. A distal portion of the probe includes a transducer positioned at a fixed location relative to the medical device in a configuration in which the probe is received by the central lumen. The medical device also includes a flushing lumen extending from the proximal end of the medical device to a distalmost opening of the flushing lumen. The flushing lumen is disposed radially outward of the central lumen, and is in fluid communication with a fluid source at the proximal end of the medical device. The distalmost opening is proximal of the fixed location such that a flow of fluid received from the fluid source and expelled by the flushing lumen via the distalmost opening at least partially radially surrounds the transducer.

### CROSS-REFERENCE TO RELATED APPLICATIONS

This application claims the benefit of priority of U.S. Provisional Patent Application No. 63/550,663, filed on Feb. 7, 2024, the entirety of which is incorporated herein by reference.

### TECHNICAL FIELD

The disclosure relates generally to systems and devices for ultrasound imaging. More specifically, aspects of the disclosure pertain to multi-lumen ultrasound imaging devices with enhanced flushing and/or articulation.

### BACKGROUND

As part of a biopsy procedure, an ultrasound imaging device, such as a radial ultrasound probe, may be first navigated into a body lumen and used to identify a target area (e.g., a suspect area) within the body lumen to be biopsied. Often, the ultrasound imaging device includes a single-lumen catheter lacking articulation, and is reusable. The single-lumen catheter may house imaging components of the ultrasound imaging device, such as a transducer configured to emit and receive sound waves for use in generating an ultrasound image.

### SUMMARY

According to some aspects, the techniques described herein relate to a medical device. An example medical device includes a central lumen and a flushing lumen. The central lumen extends from a proximal end to a distal end of the medical device, and is configured to receive a probe. A distal portion of the probe includes a transducer configured to perform ultrasound imaging, and the transducer is positioned at a fixed location relative to the medical device in a configuration in which the probe is received by the central lumen. The flushing lumen extends from the proximal end of the medical device to a distalmost opening of the flushing lumen. The flushing lumen is disposed radially outward of the central lumen, and is in fluid communication with a fluid source at the proximal end of the medical device. The distalmost opening is proximal of the fixed location such that a flow of fluid received from the fluid source and expelled by the flushing lumen via the distalmost opening at least partially radially surrounds the transducer.

In any of the example medical devices described herein, the flushing lumen is a first flushing lumen extending from the proximal end of the medical device to a first distalmost opening proximal from the fixed location, and the medical device further includes a second flushing lumen. The second flushing lumen extends from the proximal end of the medical device to a second distalmost opening of the second flushing lumen, and is disposed radially outward of the central lumen. The second flushing lumen is in fluid communication with the fluid source at the proximal end of the medical device, and the second distalmost opening is proximal from the fixed location. In some aspects, the central lumen is in fluid communication with the fluid source at the proximal end of the medical device, and the central lumen is further configured to provide a flow of fluid received from the fluid source to the distal end of the medical device through the central lumen.

In some examples, the medical device includes an outer sheath, and an inner sheath that is positioned within and extends distally from the outer sheath. The central lumen is a lumen of the inner sheath. The flushing lumen is between the inner sheath and the outer sheath.

In other examples, the medical device includes a multi-lumen catheter and a cap coupled to the multi-lumen catheter. The multi-lumen catheter includes a first portion of the central lumen and a first portion of the flushing lumen. The cap includes a second portion of the central lumen and a second portion of the flushing lumen to extend the flushing lumen to the distalmost opening. In some examples, the medical device further includes an articulation wire coupled to the cap, where the articulation wire is configured to articulate a distal portion of the medical device. In such examples, the multi-lumen catheter further includes an articulation lumen, where the articulation wire extends through the articulation lumen, and a distal end of the articulation wire extends distal to a distalmost end of the articulation lumen. Additionally, the cap further includes an articulation cavity aligned with the articulation lumen, where the articulation cavity receives the distal end of the articulation wire, and the distal end of the articulation wire is secured to the cap. In other examples, at least a distal portion of the multi-lumen catheter and the cap form an articulation region, and the distal portion of the multi-lumen catheter includes a first material that is more flexible than a second material of which a proximal portion of the multi-lumen catheter is included.

In further examples, the multi-lumen catheter and the cap coupled to the multi-lumen catheter is a first device, and the medical device further includes a second device. The second device includes a single-lumen catheter and the probe, where the probe is received within a lumen of the single-lumen catheter, and the single-lumen catheter is configured to be received by and extended through the central lumen of the first device, such that a distal portion of the single-lumen catheter including the transducer extends distally past the first device. In a configuration in which the transducer is active, the distal portion of the single-lumen catheter is positioned such that the distalmost opening of the flushing lumen of the first device is positioned at a predefined distance proximal to the transducer.

In some examples, the multi-lumen catheter is coupled to a proximal end of the cap, and the medical device further includes a single-lumen catheter coupled to and extending distally from a distal end of the cap to the distal end of the medical device. The single-lumen catheter includes a third portion of the central lumen, where, when the probe is received by the central lumen, the fixed location at which the transducer is positioned is within the single-lumen catheter.

In other examples, the medical device includes a multi-lumen catheter and an overmolded component. The multi-lumen catheter includes a first portion of the central lumen and a first portion of the flushing lumen. The overmolded component is attached distally to the multi-lumen catheter. In some examples, the overmolded component includes a proximal portion including a second portion of the central lumen and a second portion of the flushing lumen to extend the flushing lumen to the distalmost opening. The overmolded component also includes a distal portion including a third portion of the central lumen, where, when the probe is received by the central lumen, the fixed location at which the transducer is positioned is within the distal portion of the overmolded component. In other examples, an articulation wire is integrated with the multi-lumen catheter and the proximal portion of the overmolded component, where the articulation wire is configured to articulate a distal portion of the medical device.

According to other aspects, the techniques described herein relate to a medical device. An example medical device includes a proximal multi-lumen catheter.

The proximal multi-lumen catheter includes a central lumen and a flushing lumen. The flushing lumen is disposed radially outward of the central lumen, and is in fluid communication with a fluid source at a proximal end of the medical device. The example medical device also includes a distal single-lumen catheter extending the central lumen from the proximal end to a distal end of the medical device, and a cap configured to couple the proximal multi-lumen catheter to the distal single-lumen catheter, where the cap extends the flushing lumen to a distalmost opening of the flushing lumen. The example medical device further includes a probe including a transducer at a distal portion of the probe. The probe is received by and extends through the central lumen toward the distal end of the medical device. The transducer is positioned at a fixed location within the distal single-lumen catheter, where the distalmost opening of the flushing lumen is proximal of the fixed location such that a flow of fluid received from the fluid source and expelled by the flushing lumen via the distalmost opening at least partially radially surrounds the transducer.

In some examples, the cap can include a step configured to receive and secure the distal single-lumen catheter to the cap. In other examples, the cap and the distal single-lumen catheter include a proximal portion and a distal portion, respectively, of an overmolded component of the medical device. In further examples, the medical device may further include an articulation wire integrated with the proximal multi-lumen catheter and the cap.

According to further aspects, the techniques described herein relate to a medical device. An example medical device includes a multi-lumen catheter. The multi-lumen catheter includes a central lumen, and a flushing lumen disposed radially outward of the central lumen, and in fluid communication with a fluid source at a proximal end of the medical device. The example medical device also includes a cap coupled to the multi-lumen catheter, the cap extending the central lumen and the flushing lumen to a distal end of the medical device. At least a distal portion of a single-lumen catheter, separate from the medical device and having a probe including a transducer positioned at a fixed location in the distal portion of the single-lumen catheter, is configured to be removably extended through the central lumen and past the distal end of the medical device, such that a flow of fluid received from the fluid source and expelled by the flushing lumen at the distal end of the medical device at least partially radially surrounds the distal portion of the single-lumen catheter.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values+/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIGS. 1A-1C depict various views of an exemplary system having a first ultrasound imaging device.

FIGS. 2A and 2B depict various views of an articulating, multi-lumen catheter.

FIGS. 3A-3C depict various views of a cap configured to be connected to the articulating, multi-lumen catheter of FIGS. 2A and 2B to form at least a portion of an ultrasound imaging device.

FIGS. 4A-4C depict various views of a second ultrasound imaging device.

FIGS. 5A-5D depicts various views of a third ultrasound imaging device FIGS. 6A-6D depicts various views of a fourth ultrasound imaging device.

### DETAILED DESCRIPTION

As briefly described above, as part of a biopsy procedure, an ultrasound imaging device, such as a radial ultrasound probe, may be first navigated into a body lumen and used to identify a target area (e.g., a suspect area) within the body lumen to be biopsied. One example biopsy procedure may be a peripheral lung biopsy procedure, requiring access to small airways of the peripheral regions of the lungs.

Often, conventional ultrasound imaging devices include a catheter with only a single lumen. An example single-lumen catheter may house imaging components of the ultrasound imaging device, such as a probe including a transducer configured to emit and receive sound waves for use in generating ultrasound images. Sound waves, and particularly sound waves at high frequencies (e.g., 30-40 megaHertz), do not transmit or travel well through air. Therefore, when the ultrasound imaging device is capturing images in body lumens with little to no naturally flowing fluid, like the airways of the lungs, the single-lumen catheter may be connected to a fluid source to enable fluid to be flushed through the single lumen and over the transducer housed therein to help with sound wave transmission (e.g., for better coupling of the transducer with the environment of body lumens). However, once the fluid is expelled from the single-lumen catheter through a distal opening thereof, the fluid may, for example, fail to flow back up and around the portion of the catheter housing the transducer, limiting the amount of fluid available to facilitate the coupling of the transducer with the external environment of the body lumen.

Additionally, conventional ultrasound imaging devices may lack articulation mechanisms, and thus lack steerability and/or maneuverability. The lack of steerability can make navigation of the device through body lumens, and particularly smaller body lumens, like the airways of the peripheral lung regions, more challenging and time-consuming. Additionally, the lack of steerability may inhibit any adjustments of the position of the probe, and specifically the transducer, relative to the target area once the ultrasound imaging device is positioned at or near the target area.

Further, conventional ultrasound imaging devices are often reusable.

Reusable devices have to be reprocessed between medical procedures to clean and/or sterilize the devices, which may be costly both in time and money. For example, the reprocessing time may cause delays between procedures, and nonetheless, reuse may still present cross-contamination risks to patients. Further, the reusable ultrasound imaging device may be prone to damage or breakage, particularly when subjected to the reprocessing.

Therefore, aspects of this disclosure are directed to multi-lumen devices for ultrasound imaging to provide enhanced fluid flushing. Additionally, at least some embodiments of the multi-lumen devices may include articulation mechanisms. Further, the multi-lumen devices may be single use or disposable devices.

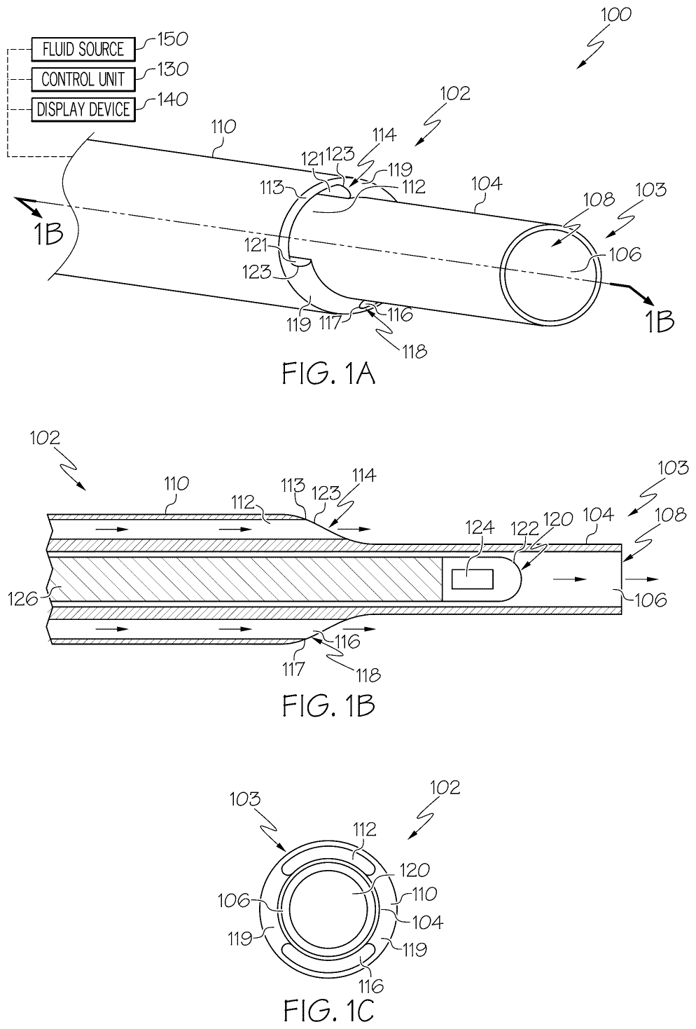

One example device, described with reference to FIGS. 1A-1C below, may be a non-articulating, multi-lumen device comprised of an inner sheath and an outer sheath. The non-articulating, multi-lumen device may be single use or disposable. An inner diameter of the inner sheath may form a central lumen configured to receive a probe, including a transducer. In some examples, the central lumen may also be in fluid communication with a fluid source to enable fluid to flow through the central lumen and over the transducer. The inner sheath may be positioned within and extend distally from the outer sheath to form one or more flushing lumens between the inner sheath and the outer sheath. Terminating ends of the flushing lumens at a distal end of the outer sheath may include distal openings that are positioned proximal of a portion of the inner sheath housing at least the transducer (e.g., an imaging window). The proximal positioning of the distal openings may help to ensure that fluid provided via the flushing lumens flushes out (e.g., replaces) air, such that the fluid at least partially radially surrounds the portion of inner sheath including the transducer to supplement and enhance effects of the fluid provided via the central lumen for improved sound wave transmission.

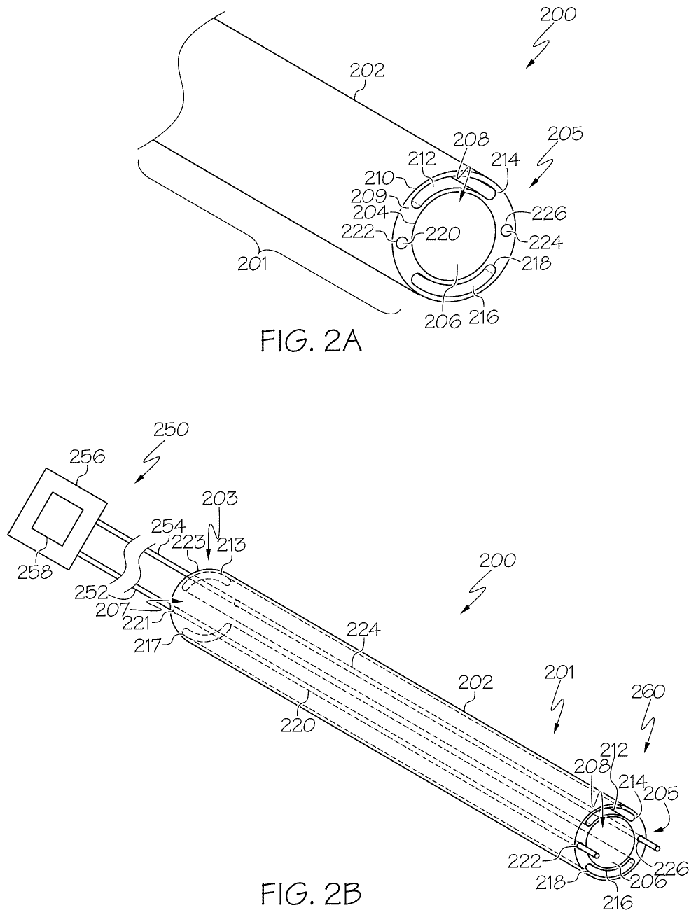

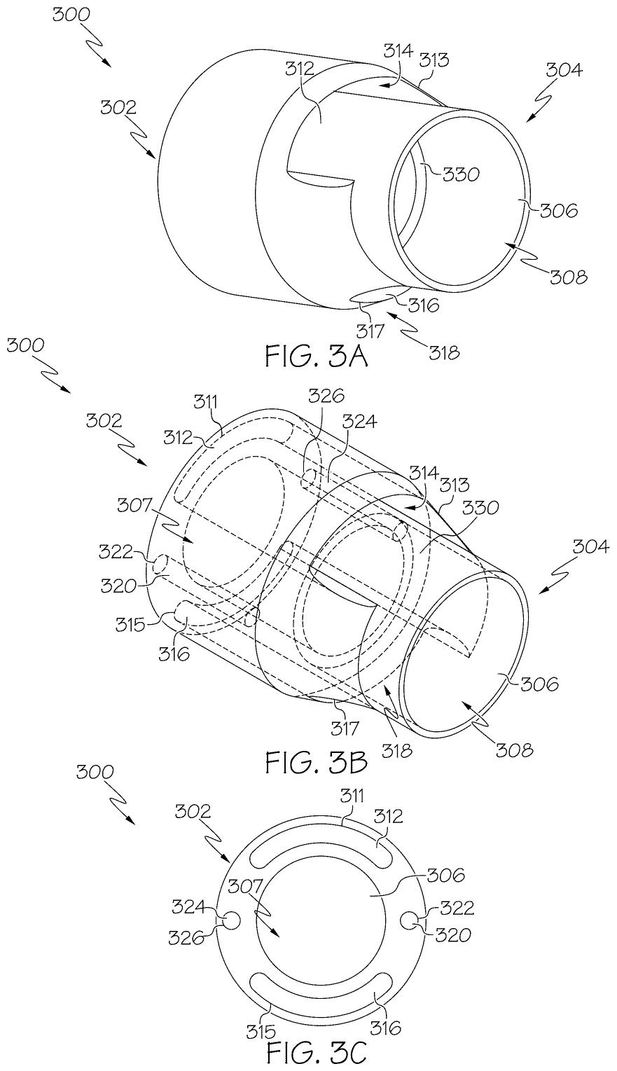

Other example devices may include articulating, multi-lumen devices. These articulating, multi-lumen devices may also be single use or disposable. Each of the articulating, multi-lumen devices may include at least a multi-lumen catheter and a cap integrated with an articulation mechanism, as shown in FIGS. 2A-B and FIGS. 3A-3C. The multi-lumen catheter may be coupled to the cap, and each of the cap and the multi-lumen catheter may include corresponding central lumens (e.g., forming a continuous central lumen), and one or more corresponding flushing lumens (e.g., forming continuous flushing lumens). The continuous central lumen may be configured to receive a probe, where a distal portion of the probe including the transducer may extend through and distally past the continuous central lumen. Terminating ends of the flushing lumens may include distal openings that are positioned proximal of a portion of the articulating, multi-lumen device housing the transducer (e.g., an imaging window) for similar reasons described above with respect to the non-articulating, lumen device.

Additionally, the multi-lumen catheter may include articulation lumens configured to receive distal ends of one or more articulation wires of the articulation mechanism. The distal ends of the articulation wires may extend past the multi-lumen catheter and into one or more articulation cavities of the cap, where the articulation wires may be secured to a wall of the articulation cavities. The articulation mechanism may further include one or more actuators to which proximal ends of the articulation wires may be connected. Upon receiving an operator action, the actuators may be configured to actuate the articulation wires to enable steerability of a distal portion of the device (e.g., an articulation region where the multi-lumen catheter is coupled to the cap). The steerability provided by the articulation mechanism may facilitate navigation of the articulating, multi-lumen device through body lumens. Further, when the articulating, multi-lumen device is positioned near an intended target, steerability of the articulation region provided by the articulation mechanism may enable adjustment of a position of the distal portion of the probe, and specifically the transducer, relative to the target area to help improve image quality.

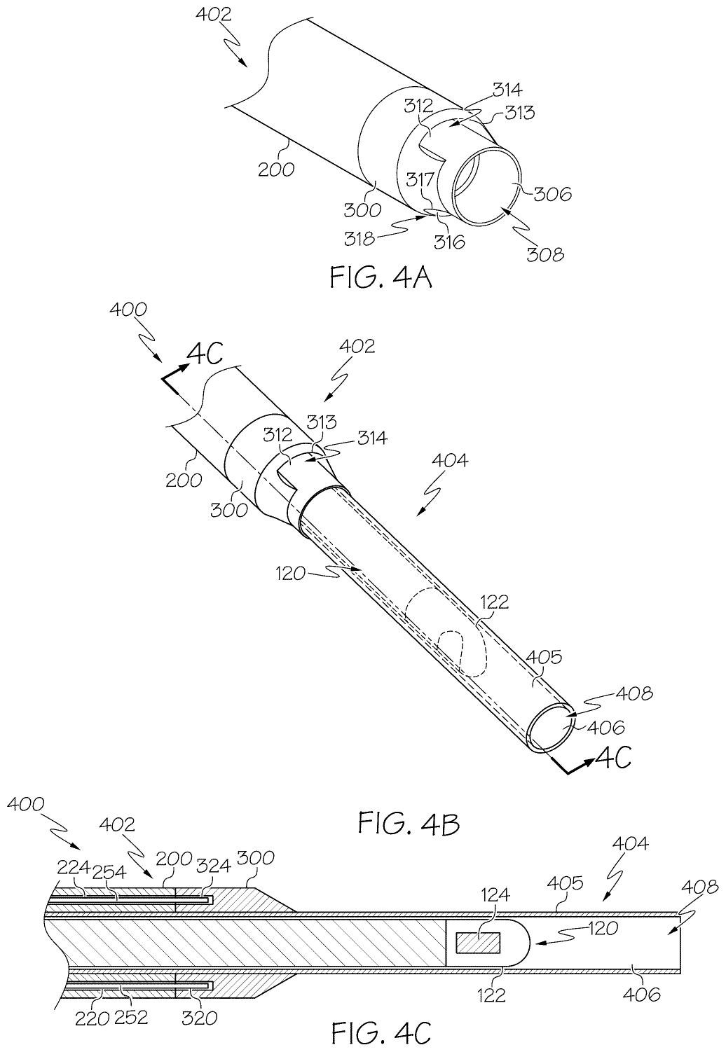

One example articulating, multi-lumen device, described with reference to FIGS. 4A-4C, may include a first device comprised of the multi-lumen catheter coupled to the cap, and a second device comprised of a single-lumen catheter (e.g., the imaging window) including the probe received within the single lumen of the catheter. The second device may be inserted into and extended distally past a distal end of the first device, such that the distal openings at the terminating ends of the flushing lumens of the first device are positioned proximal from at least a distal portion of the second device housing the transducer.

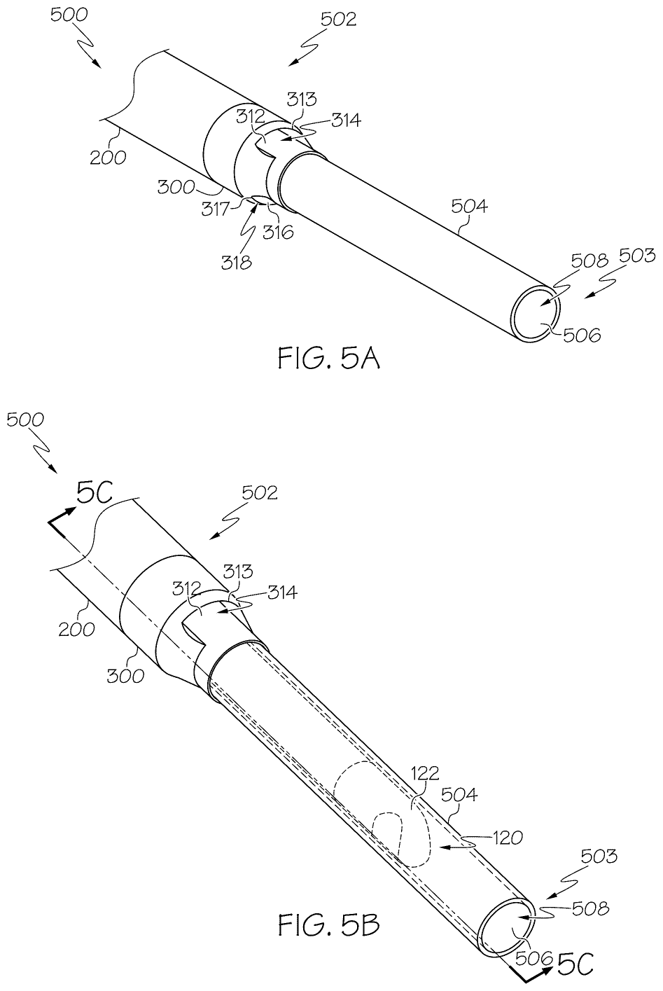

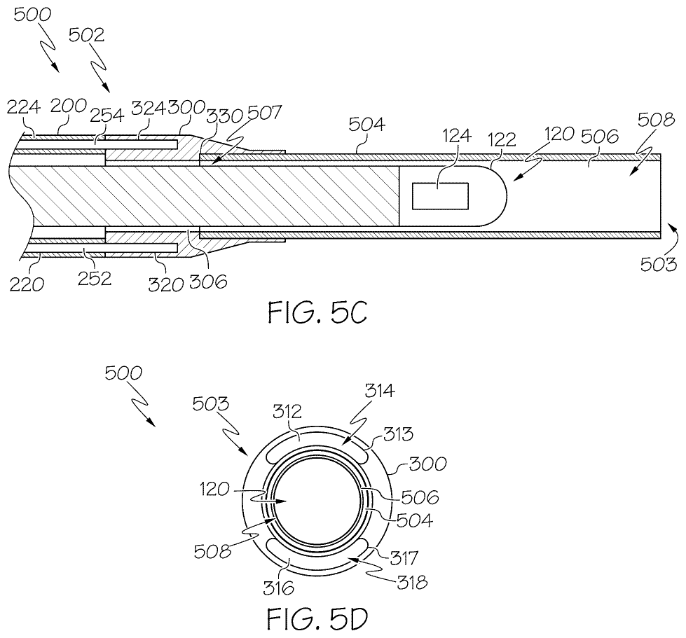

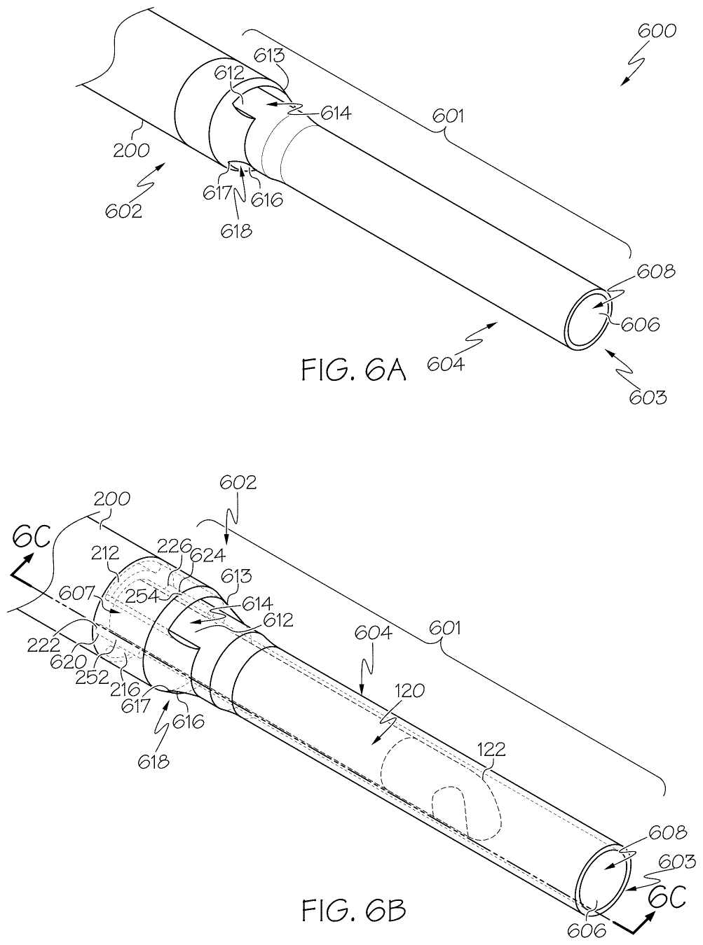

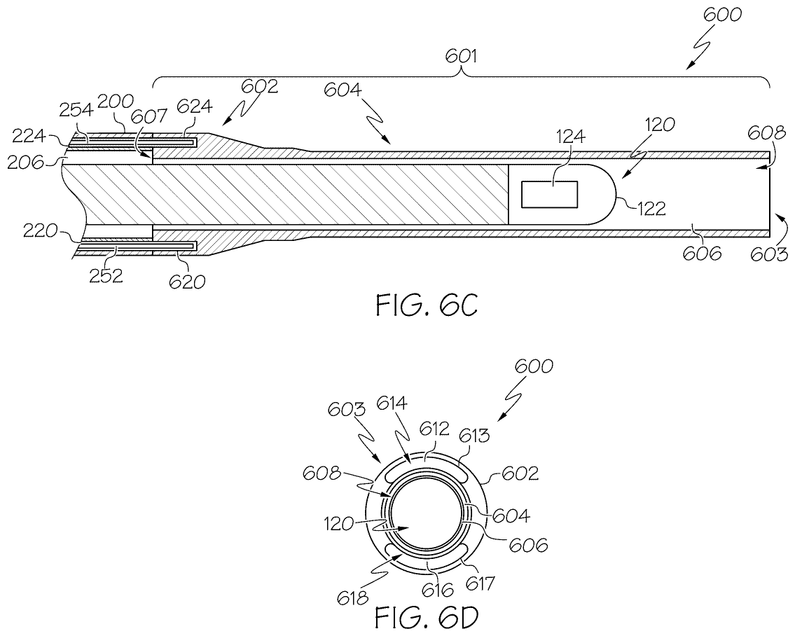

Another example articulating, multi-lumen device, described with reference to FIGS. 5A-5D, may include a single device having an articulating, multi-lumen component attached proximally to a single-lumen catheter (e.g., the imaging window). The articulating, multi-lumen component may include the multi-lumen catheter coupled to the cap. The single-lumen catheter may be received by and attached to the cap. The single-lumen catheter may be configured to receive at least a distal portion of the probe, including the transducer, at a fixed location, such that the distal openings at the terminating ends of the flushing lumens of the articulating, multi-lumen component are positioned proximal to the transducer.

A further example articulating, multi-lumen device, described with reference to FIGS. 6A-6D, may include a single device having the multi-lumen catheter attached proximally to an overmolded component. The overmolded component may include a proximal portion comprised of the cap and/or features of the cap, and a distal portion in a form of a single-lumen catheter (e.g., the imaging window). The distal portion of the overmolded component may be configured to receive at least a distal portion of the probe, including the transducer, at a fixed location within the lumen thereof, such that the distal openings at the terminating ends of the flushing lumens (e.g., located at a distal end of the proximal portion) are positioned proximal to the transducer.

The specific examples included throughout the present disclosure implement an ultrasound imaging system, and more specifically, a radial ultrasound imaging system configured to perform radial ultrasound imaging within airways of the peripheral lung regions. However, it should be understood that techniques according to this disclosure may be adapted to other ultrasound imaging systems used to image other body lumens, and particularly body lumens with limited or no natural fluid flow and/or content. It should also be understood that the examples above are illustrative only. The techniques and technologies of this disclosure may be adapted to any suitable activity.

Referring now to the figures, FIG. 1A depicts an exemplary system 100 having a first ultrasound imaging device 102, hereinafter device 102 for brevity. System 100 may also include a control unit 130, a display device 140, and/or a fluid source 150. Device 102 may be a medical device, and specifically a non-articulating, ultrasound imaging device, that may be removably connected to control unit 130, display device 140, and/or fluid source 150 to perform ultrasound imaging during diagnostic and/or interventional medical procedures. Device 102 may be a disposable or single use device such that device 102 is removed from system 100 and discarded or disposed of after completion of a medical procedure.

One example medical procedure may include a peripheral lung biopsy, in which ultrasound imaging performed by system 100 may be utilized to identify a target area (e.g., a suspicious area) within an airway of the peripheral regions of the lung to sample tissue from. For example, a distal portion of device 102 may be positioned within and navigated through an airway in a peripheral region of the lung expected to contain a lesion or other similar abnormality. Once the target area has been identified utilizing the ultrasound imaging provided by system 100, device 102 may be removed from the airway and disposed. In some examples, a biopsy device and/or an ultrasound-guided biopsy device may be positioned at the location of the identified target area prior to or after removal of device 102 to collect a sample of the target area.

FIG. 1A includes a perspective view of the distal portion of device 102. FIG. 1B depicts a cross-sectional view of device 102 taken along line 1B in FIG. 1A. FIG. 1C depicts a front view of a distal end 103 of device 102. Referring concurrently to FIGS. 1A-1C, device 102 may include an inner sheath 104 and an outer sheath 110. Inner sheath 104 and outer sheath 110 may form a non-articulating, multi-lumen catheter. In some examples, inner sheath 104 and outer sheath 110 may be fixed relative to one another. For example, inner sheath 104 and outer sheath 110 may be axially fixed and/or rotationally fixed to one another. Additionally, while inner sheath 104 and outer sheath 110 are described as separate sheaths joined to one another to form device 102, in other examples, device 102 may be comprised of a singular sheath (e.g., a single multi-lumen, extruded sheath) having features of inner sheath 104 and outer sheath 110. Inner sheath 104 and/or outer sheath 110 may be composed of one or a combination of varying types of plastic materials, including both thermoplastics and thermosets, such as PEBAX®, Nylon, Polytetrafluoroethylene (PTFE), and Expanded PTFE. In some examples, at least outer sheath 110 may be in a form of a composite coil or braid for improved torque and/or rotation flexibility.

Inner sheath 104 may include a central lumen 106 extending from a proximal end (not shown) of device 102 to a distal end 103 of device 102. A distal opening 108 of central lumen 106 may be positioned at distal end 103, and may be distal facing. Specifically, distal opening 108 may be positioned at a distalmost end of distal end 103 of device 102.

Device 102 may also include a probe 120. As shown in FIG. 1B, central lumen 106 may be configured to receive or house probe 120. Probe 120 may be an ultrasound probe. In some examples, probe 120 may be a radial ultrasound probe. A distal portion 122 of probe 120 may include a transducer 124. Transducer 124 may be configured to receive electrical signals generated by control unit 130, and transform the electrical signals into mechanical sound waves. Transducer 124 may emit the sound waves into the surrounding environment (e.g., an airway within a peripheral lung region), and the sound waves may reflect off of internal structures within the environment, and return to transducer 124. Transducer 124 may then convert the returned sound waves into electrical signals, which are transmitted to control unit 130 for processing (e.g., to generate an ultrasound image). Control unit 130 may then provide the ultrasound image to display device 140 for display. While only one transducer (e.g., transducer 124) is shown in FIG. 1B, in other examples, distal portion 122 of probe 120 may include a plurality of transducers.

Distal portion 122 of probe 120, including transducer 124, may be positioned at a fixed location within a distal portion of central lumen 106, as described in further detail below. The distal portion of central lumen 106 in which transducer 124 is positioned may be also referred to herein as a visualization region or imaging window. A proximal portion of probe 120 may include a shaft 126. Shaft 126 may house connection elements (not shown), such as cables, wires, etc., extending from control unit 130 to transducer 124 to enable communication of the above-described electrical signals therebetween.

Sound waves, and particularly sound waves at high frequencies (e.g., 30-40 megaHertz), do not transmit or travel well through air. Fluid, such as water or any other similar flushing solution, may need to be flushed over (e.g., such that the fluid pushes out the air and surrounds) transducer 124 as device 102 is being operated to help with sound wave transmission to ensure optimal image quality. Such flushing may be critical when system 100 is being used for ultrasound imaging in body lumen environments where the body lumen naturally contains little to no fluid, such as in the airways of the peripheral lung regions. Accordingly, a proximal end (not shown) of central lumen 106 may be configured to be mechanically and fluidly connected to fluid source 150 to establish a fluid communication between central lumen 106 and fluid source 150.

Fluid source 150 may include a reservoir containing the fluid to be used for flushing. In some examples, fluid source 150 may include one or more syringes containing the fluid, such as water, for flushing. The syringe(s) may be pre-filled with a predetermined volume of fluid. A syringe may be mechanically coupled to (e.g. inserted into) a respective lumen of device 102, such as central lumen 106. An operator may actuate the syringe to expel the fluid from the syringe by, for example, pressing down on a plunger of the syringe. As the fluid flows distally through central lumen 106, at least a portion of the fluid may remain within the lumens due to surface tension and/or capillary action for example.

In other examples, fluid source 150 may be an automated fluid delivery device that is communicatively coupled to control unit 130 via wired or wireless connections such that fluid source 150 may receive instructions or signals from control unit 130. Fluid source 150 may be a separate device from or integrated with (e.g., a component of) control unit 130. Example instructions or signals received from control unit 130 may cause fluid source 150 to initiate and subsequently terminate an expelling of at least a portion of fluid within the reservoir to device 102. Resultantly, when central lumen 106 is in fluid communication with fluid source 150, and fluid source 150 expels the fluid in response to instructions received from control unit 130, the fluid may flow, via central lumen 106, to distal end 103 of device 102 and out of distal opening 108 (e.g., as depicted by the arrows in FIG. 1B).

The flow of fluid through central lumen 106 enables the flow of fluid over probe 120, positioned within central lumen 106, as the fluid is expelled toward and out of distal opening 108. However, once the fluid is expelled from distal opening 108, the fluid may, for example, fail to flow proximally back up and around distal portion of device 102 and thus transducer 124. Resultantly, providing fluid flushing via only central lumen 106 may be insufficient to ensure constant fluid flow surrounding transducer 124 to facilitate sound wave transmission.

Therefore, in addition to central lumen 106, device 102 may include one or more flushing lumens, such as a first flushing lumen 112 and a second flushing lumen 116, that are disposed radially outward of central lumen 106. For example, inner sheath 104 may be positioned within outer sheath 110 to form first flushing lumen 112 and second flushing lumen 116 between inner sheath 104 and outer sheath 110. In some examples, first flushing lumen 112 and second flushing lumen 116 each may extend parallel to a central longitudinal axis of device 102, such that cross-sections of, first flushing lumen 112 and second flushing lumen 116 may be parallel to one another, as shown in FIG. 1B.

First flushing lumen 112 may extend from a first proximal end (not shown) at the proximal end of device 102 to a first terminating end 113. First terminating end 113 may be proximal to distal end 103 of device 102 because inner sheath 104 may extend distally past a distalmost end of outer sheath 110. First terminating end 113 may be a distalmost end of outer sheath 110, and may include a first distal opening 114 that is distal-facing. First distal opening 114 may be a distalmost opening of first flushing lumen 112. First distal opening 114 may be proximal of the fixed location at which the distal portion 122 of probe 120 is positioned with the distal portion of central lumen 106 (e.g., proximal from the visualization region or imaging window). For example, first distal opening 114 may be a first predefined distance proximal to transducer 124.

Second flushing lumen 116 may extend from a second proximal end (not shown) at the proximal end of device 102 to a second terminating end 117 proximal to distal end 103 of device 102 (because, as discussed above, inner sheath 104 may extend distally past a distalmost end of outer sheath 110). Second terminating end 117 may be a distal end of outer sheath 110, and may include a second distal opening 118 that is distal-facing. Second distal opening 118 may be a distalmost opening of second flushing lumen 116. Second distal opening 118 may be proximal from the fixed location at which distal portion 122 of probe 120 is positioned with the distal portion of central lumen 106 (e.g., proximal from the visualization region or imaging window). For example, second distal opening 118 may be a second predefined distance proximal to transducer 124. In some examples, the first distal opening 114 and second distal opening 118 may be the same predefined distance proximal to transducer 124 (e.g., the first and second predefined distances may be substantially equal). In other words, first distal opening 114 and second distal opening 118 may be at a same axial position along device 102.

As shown in FIG. 1A, outer sheath 110 may include protruding portions 119 (e.g., two protruding portions 119) between first flushing lumen 112 and second flushing lumen 116. Protruding portions 119 may protrude radially inward from outer sheath 110 to inner sheath 104 in order to separate first flushing lumen 112 from second flushing lumen 116. In some examples, radially inner surfaces of protruding portions 119 may be fixedly coupled to inner sheath 104 or may otherwise be sealed with respect to inner sheath 104 to help to retain fluid within first flushing lumen 112 and second flushing lumen 116. Surfaces 121 of protruding portions 119 may define sides of first flushing lumen 112 and second flushing lumen 116. In aspects, distal ends of protruding portions 119 may be distal to portions or entireties of first terminating end 113 and second terminating end 117. In some examples, and as most clearly shown in FIG. 1B, edges 123 between surfaces 121 of protruding portions 119 and distal-facing surfaces of protruding portions 119 may be sloped or angled distally relative to (e.g., as opposed to perpendicular to) a central longitudinal axis of central lumen 106.

Each of the first proximal end of first flushing lumen 112 and the second proximal end of second flushing lumen 116 may be configured to be mechanically and fluidly connected to fluid source 150 to establish a fluid communication between flushing lumens 112, 116 and fluid source 150. When first flushing lumen 112 is in fluid communication with fluid source 150, and fluid source 150 provides the fluid (e.g., based on a manual actuation or in response to instructions received from control unit 130 if automated), the fluid may flow through first flushing lumen 112 to first terminating end 113 and out of first distal opening 114 (e.g., as depicted by the arrows in FIG. 1B). Similarly, when second flushing lumen 116 is in fluid communication with fluid source 150, and fluid source 150 provides the fluid (e.g., based on a manual actuation or in response to instructions received from control unit 130 if automated), the fluid may flow through second flushing lumen 116 to second terminating end 117 and out of second distal opening 118 (e.g., as depicted by the arrows in FIG. 1B).

In some examples, each of central lumen 106, first flushing lumen 112, and second flushing lumen 116 may establish a fluid communication with fluid source 150 simultaneously when proximal end of device 102 is mechanically connected to fluid source 150, for example. Therefore, when fluid source 150 provides the fluid based on a manual actuation or in response to instructions received from control unit 130 if automated, fluid may be simultaneously provided through central lumen 106, first flushing lumen 112, and second flushing lumen 116. In other examples, central lumen 106, first flushing lumen 112, and/or second flushing lumen 116 may establish a fluid communication with fluid source 150 independently of one another. To provide an example, when fluid source 150 comprises one or more syringes, a single syringe may be mechanically and fluidly connected to each of central lumen 106, first flushing lumen 112, and second flushing lumen 116 simultaneously to enable concurrent flushing of fluid through each of central lumen 106, first flushing lumen 112, and second flushing lumen 116 upon actuation of the syringe. Alternatively, a different syringe (e.g., a first syringe, a second syringe, and a third syringe) may be mechanically and fluidly connected to each of central lumen 106, first flushing lumen 112, and second flushing lumen 116 to enable independent flushing of fluid through each of central lumen 106, first flushing lumen 112, and second flushing lumen 116 upon actuation of the respective syringe.

The position of first terminating end 113 and second terminating end 117 proximal to the fixed location of distal portion 122 of probe 120 within central lumen 106 of inner sheath 104 helps to ensure that the fluid expelled from first flushing lumen 112 and second flushing lumen 116 via first distal opening 114 and second distal opening 118 at least partially radially flushes out (e.g., replaces) air and surrounds the portion of inner sheath 104 including transducer 124 (e.g., surrounds the visualization region or imaging window). The fluid provided via first flushing lumen 112 and second flushing lumen 116 may supplement and enhance the fluid provided via central lumen 106 for improved sound wave transmission.

While two flushing lumens are shown in FIGS. 1A-1C, in other examples, device 102 may include fewer or more flushing lumens. For example, device 102 may alternatively include only a single flushing lumen that is disposed radially outward of central lumen 106. As another example, device 102 may alternatively include three or more flushing lumens that each at least partially surround inner sheath 104.

Control unit 130 may be a computing system, a computing device, a controller, or other similar standalone processing unit. Control unit 130 may include at least one memory and at least one processor. The memory may be configured to store instructions to be executed by the processor to cause control unit 130 to perform corresponding operations, including but not limited to: transmitting instructions and/or signals to fluid source 150 (e.g., if automated) to cause fluid to be provided from fluid source 150 to device 102, generating and transmitting electrical signals to device 102, receiving return electrical signals from device 102, processing the return electrical signals to generate images, and causing display of the images and/or other data (e.g., via display device 140). The memory may also include one or more data stores. Additionally or alternatively, control unit 130 may include one or more data stores separate from the memory.

Display device 140 may be configured to display data associated with one or more of device 102, control unit 130, and/or fluid source 150. In some examples, displayed data may include images generated by control unit 130 based on signals received from device 102. Additionally, displayed data may indicate whether fluid is currently being supplied from fluid source 150 to device 102, an amount of fluid supplied, and/or an amount of fluid remaining in the reservoir of fluid source 150, among other similar examples. Display device 140 may include one or more a combination of monitors, computing device screens, touch screen display devices, etc. In some examples, display device 140 may be a separate device from control unit 130 that is communicatively coupleable to control unit 130 via wired and/or wireless connections. In other examples, display device 140 may be a display or screen of control unit 130 itself.

In some examples, control unit 130 may generate, or may cause to be generated, one or more graphical user interfaces based on instructions or information stored in the memory of control unit 130, instructions or information received from device 102, instructions or information received from fluid source 150, and/or the like and may cause the graphical user interfaces to be displayed via display device 140. The graphical user interfaces may include text, visual elements, controls, and/or the like, in addition to the displayed data. Display device 140 may include a touch screen or a display with other input systems (e.g., a mouse, keyboard, voice, etc.) for an operator of control unit 130 to control functions of control unit 130, device 102 and/or fluid source 150 via control unit 130 and/or display device 140.

In some aspects, one or more components of system 100, such as device 102, control unit 130, display device 140, and/or fluid source 150, may be capable of network connectivity, and may communicate with one another over a wired network or a wireless network. The network may be an electronic network. The network may include a wide area network (“WAN”), a local area network (“LAN”), personal area network (“PAN”), a cellular network (e.g., a 3G network, a 4G network, a 5G network, etc.), or the like. In other examples, the components of system 100 may communicate and/or connect to the network over universal serial bus (USB) or other similar local, low latency connections or direct wireless protocol. Components of system 100 may be connected via the network, using one or more standard communication protocols, such that the component may transmit and receive communications from each other across the network.

Although various components in system 100 are depicted as separate components in FIG. 1A, it should be understood that a component or portion of a component in system 100 may, in some embodiments, be integrated with or incorporated into one or more other components. For example, display device 140 and/or fluid source 150 may be integrated with control unit 130. In some embodiments, operations or aspects of one or more of the components discussed above may be distributed amongst one or more other components. Any suitable arrangement and/or integration of the various systems and devices of system 100 may be used.

As shown and described with reference to FIGS. 1A-1C, inner sheath 104 and outer sheath 110 of device 102 form a non-articulating, multi-lumen catheter of system 100 that is configured to receive probe 120. FIGS. 2A and 2B depict a multi-lumen catheter 200, hereinafter catheter 200, that is integrated with an articulation mechanism 250 and configured to receive probe 120. FIGS. 3A-3C depict various views of a cap 300 configured to be connected to a distal end of catheter 200 to form at least a portion of a medical device, and specifically an articulating ultrasound imaging device, such as second ultrasound imaging device 400 described with reference to FIGS. 4A-C and/or a third ultrasound imaging device 500 described with reference to FIGS. 5A-D. The articulating ultrasound imaging device may replace (e.g., may be used instead of or in place of) device 102 in system 100. For example, the articulating ultrasound imaging device may be electrically and/or mechanically connected to control unit 130, display device 140, and/or fluid source 150. Similar to device 102, the articulating ultrasound imaging device may be a disposable or single use device.

FIG. 2A depicts a perspective view of a distal portion 201 of catheter 200. FIG. 2B depicts articulation mechanism 250 integrated with catheter 200. Catheter 200 is transparent in FIG. 2B to depict how articulation mechanism 250 is integrated. Broken lines in FIG. 2B and other figures herein depict structures that are internal to other structures (e.g., catheter 200 in FIG. 2B). For example, articulation mechanism 250 may include one or more articulation wires, such as a first articulation wire 252 and a second articulation wire 254 that are received by and extend through and distally past catheter 200. Articulation mechanism 250 may also include an actuator 258 to actuate first articulation wire 252 and second articulation wire 254. In some examples, actuator 258 may be disposed on a handle 256 to facilitate operation of the actuator 258 by an operator.

Referring now concurrently to FIGS. 2A and 2B, catheter 200 may include a tubular body or tube 202. Tube 202 may be composed of one or a combination of varying types of plastic materials, including both thermoplastics and thermosets, such as PEBAX®, Nylon, Polytetrafluoroethylene (PTFE), and Expanded PTFE. In some examples, tube 202 may be in a form of a composite coil or braid for improved torque and/or rotation flexibility. While tube 202 may have some flexibility, tube 202 may also be sufficiently rigid to enable maneuverability of catheter 200 through body lumens, including airways in the peripheral lung region.

Tube 202 may include an inner diameter 204 forming a central lumen 206 Central lumen 206 may include a proximal opening 207 at proximal end 203 of tube 202 and a distal opening 208 at distal end 205 of tube 202. Central lumen 206 of catheter 200 may be similar to central lumen 106 of device 102. For example, central lumen 206 may be configured to receive probe 120. However, and as discussed in greater detail below, central lumen 206 may only be a first portion of a central lumen of a given ultrasound imaging device, and distal portion 122 of probe 120 that includes transducer 124 may be extended through central lumen 206 and positioned at a fixed location distal to central lumen 206.

Additionally, a proximal opening 207 of central lumen 206 may be configured to be mechanically connected to fluid source 150 to establish a fluid communication. Flow of fluid from fluid source 150 through central lumen 206 may help promote flow of fluid over probe 120 (not shown in FIGS. 2A and 2B but shown in FIGS. 1A-1C) to facilitate sound wave transmission by transducer 124. However, for the same or similar reasons discussed above with reference to FIGS. 1A-1C, providing fluid flushing via only central lumen 206 may be insufficient to ensure sufficient fluid flow surrounding transducer 124. Therefore, in addition to central lumen 206, tube 202 may also include one or more flushing lumens, such as first flushing lumen 212 and second flushing lumen 216, that are disposed radially outward of central lumen 206.

For example, tube 202 may include a wall 209 extending from inner diameter 204 to an outer diameter 210 of tube 202. First flushing lumen 212 and second flushing lumen 216 may extend within wall 209 from proximal end 203 to distal end 205 of tube 202. In some examples, first flushing lumen 212 and second flushing lumen 216 may each extend parallel to a central longitudinal axis of tube 202 and may be diametrically opposed to one another across tube 202. At proximal end 203 of tube 202, wall 209 may include a first proximal wall opening 213 of first flushing lumen 212 and a second proximal wall opening 217 of second flushing lumen 216. First proximal wall opening 213 and second proximal wall opening 217 may establish a fluid connection between tube 202 and fluid source 150 such that fluid provided from fluid source 150 may be received by and flow distally through first flushing lumen 212 and second flushing lumen 216, respectively. At distal end 205 of tube 202, wall 209 may include a first distal wall opening 214 of first flushing lumen 212 and a second distal wall opening 218 of second flushing lumen 216. First distal wall opening 214 and second distal wall opening 218 may be configured to provide fluid from tube 202 to cap 300, as described in more detail with reference to FIGS. 3A-3C below.

Additionally, tube 202 may include one or more articulation lumens, such as first articulation lumen 220 and second articulation lumen 224, that extend within wall 209 from proximal end 203 to distal end 205 of tube 202. At proximal end 203 of tube 202, wall 209 may include a third proximal wall opening 221 of first articulation lumen 220 and a fourth proximal wall opening 223 of second articulation lumen 224. At distal end 205 of tube 202, wall 209 may include a third distal wall opening 222 of first articulation lumen 220 and a fourth distal wall opening 226 of second articulation lumen 224. To integrate articulation mechanism 250 with catheter 200, first articulation lumen 220 and second articulation lumen 224 may receive distal ends of first articulation wire 252 and second articulation wire 254 via third proximal wall opening 221 and fourth proximal wall opening 223, respectively, at proximal end 203 of tube 202. Distal ends of first articulation wire 252 and second articulation wire 254 may then be extended through and distally past first articulation lumen 220 and second articulation lumen 224 via third distal wall opening 222 and fourth distal wall opening 226, respectively. Distal ends of first articulation wire 252 and second articulation wire 254 may then be received by and secured to cap 300, as described in more detail with reference to FIGS. 3A-3C below.

As previously mentioned, tube 202 may be composed of one or a combination of varying types of plastic materials. In some examples, at least distal end 205 of tube 202 forming an articulation region (e.g., along with cap 300) may be composed of a first material, whereas a proximal portion of tube 202 may be composed of a second material. The first material may be more flexible than the second material. For example, the first material may be Expanded PTFE, and the second material may be PTFE. Expanded PTFE may be PTFE that has been expanded through a linear retraction process. Expanded PTFE has a lower density than PTFE. Lower density provides greater flexibility. Therefore, expanded PTFE at the articulation region may provide a controlled region with increased flexibility for navigation and articulation of tube 202.

Proximal ends of first articulation wire 252 and second articulation wire 254 may be attached to actuator 258. In some examples, actuator 258 may be a lever mechanism that can be moved or pushed upward or downward to actuate first articulation wire 252 and second articulation wire 254. Additionally or alternatively, actuator 258 may include a sliding mechanism, a rotating mechanism (e.g., a knob), a pushing mechanism, or the like to actuate first articulation wire 252 and second articulation wire 254. Actuation of first articulation wire 252 and second articulation wire 254 may cause an articulation region 260 (e.g., a region including where catheter 200 couples to cap 300 at distal portion 201) to articulate or move along a first directional plane (e.g., move right and left or up and down).

FIGS. 2A and 2B show articulation mechanism 250 as having two articulation wires and one actuator. However, in other examples, articulation mechanism 250 may have more or fewer articulation wires. Additionally, in examples where articulation mechanism 250 may include more articulation wires, articulation mechanism 250 may include at least two actuators to allow movement of articulation region 260 in at least two directions (e.g., up and down and left and right). For example, articulation mechanism 250 may include four articulation wires, where two articulation wires may be attached to a first actuator, and two articulation wires may be attached to a second actuator.

Further, when articulation mechanism 250 may have more or fewer articulation wires, tube 202 may have more or fewer articulation lumens for receiving the articulation wires. First flushing lumen 212 and second flushing lumen 216 may be configured, positioned, and/or arranged alternatively based on a number and corresponding positions of the articulation lumens for receiving the articulation wires. For example, if articulation mechanism 250 includes four articulation lumens to receive four articulation wires, the articulation lumens may be positioned perpendicular to (90 degrees from) one another within wall 209 of tube 202. In one example configuration, each of first flushing lumen 212 and second flushing lumen 216 may be separated into two portions by one of the articulation lumens (e.g., resulting in four flushing lumens total). In another example configuration, first flushing lumen 212 and second flushing lumen 216 may be configured such that first flushing lumen 212 and second flushing lumen 216 are arranged or shaped around or between the articulation lumens. Any configurations and/or arrangements of flushing lumens, such as first flushing lumen 212 and second flushing lumen 216, in which the flushing lumens are disposed radially outward of (e.g., at least partially surround) central lumen 206 may be utilized.

FIGS. 3A-3C depict various views of cap 300 configured to be connected to distal portion 201 of catheter 200. For example, FIG. 3A depicts a perspective view of cap 300. FIG. 3B depicts a transparent perspective view of cap 300, with broken lines indicating internal structures of cap 300 or structures that are not otherwise visible on an outside of cap 300 in the view of FIG. 3B. FIG. 3C depicts a planar view of a proximal end 302 of cap 300. Cap 300 may be composed of plastic, metal, or other similar materials. Cap 300 may be formed using three-dimensional (3-D) printing or may be machine manufactured.

Referring to FIGS. 3A-3C concurrently, cap 300 may include a central lumen 306 extending from proximal end 302 of cap 300 to a distal end 304 of cap 300. Central lumen 306 may include a proximal opening 307 at proximal end 302 and a distal opening 308 at distal end 304. A diameter of proximal opening 307 may correspond to inner diameter 204 of tube 202 of catheter 200 such that, when cap 300 is connected distally to catheter 200, central lumen 306 of cap 300 extends central lumen 206 of catheter 200 to form a continuous lumen that is configured to receive probe 120 and fluid flow from fluid source 150. In other words, central lumen 206 of catheter 200 may be a first portion of the continuous central lumen, and central lumen 306 of cap 300 may be a second portion of the continuous central lumen distal from the first portion. Resultantly, fluid from fluid source 150 may flow through central lumen 206 of tube 202 into central lumen 306 via proximal opening 307, and out of central lumen 306 via distal opening 308. In some aspects, the continuous lumen may be extended even further via single-lumen catheters distally coupled to or otherwise integrated with cap 300 (e.g., forming a third portion of the continuous central lumen distal from the second portion), as described in more detail below.

Cap 300 may include one or more flushing lumens, including a first flushing lumen 312 and a second flushing lumen 316, extending from proximal end 302 to terminating end proximal of distal end 304 of cap 300. For example, first flushing lumen 312 of cap 300 may correspond to (be aligned with) first flushing lumen 212 of tube 202 to extend first flushing lumen 212 to a first terminating end 313. In other words, first flushing lumen 212 of tube 202 and first flushing lumen 312 of cap 300 may form a continuous first flushing lumen that extends from the proximal end 203 of tube 202 (FIG. 2B) to first terminating end 313. For example, a first proximal opening 311 of first flushing lumen 312 on a proximal-facing surface of proximal end 302 of cap 300 may correspond to (be aligned with) first distal wall opening 214 of first flushing lumen 212 of tube 202. A first distal opening 314 of first flushing lumen 312 may be located at first terminating end 313 to allow fluid received from fluid source 150 via first flushing lumen 212 and first flushing lumen 312 to be expelled at first terminating end 313. First distal opening 314 may be a distalmost opening of the continuous first flushing lumen.

Similarly, second flushing lumen 316 of cap 300 may correspond to (be aligned with) second flushing lumen 216 of tube 202 to extend second flushing lumen 216 to a second terminating end 317. In other words, second flushing lumen 216 of tube 202 and second flushing lumen 316 of cap 300 may form a continuous second flushing lumen that extends from the proximal end 203 of tube 202 (FIG. 2B) to second terminating end 317. For example, a second proximal opening 315 of second flushing lumen 316 on the proximal-facing surface of proximal end 302 of cap 300 may correspond to (be aligned with) second distal wall opening 218 of second flushing lumen 216 of tube 202. A second distal opening 318 of second flushing lumen 316 may be located at second terminating end 317 to allow fluid received from fluid source 150 via second flushing lumen 216 and second flushing lumen 316 to be expelled at second terminating end 317. Second distal opening 318 may be a distalmost opening of the continuous second flushing lumen.

In some examples, and as shown in FIGS. 3A-3B, portions of cap 300 that are adjacent to first terminating end 313 and second terminating end 317 may be sloped or angled inward toward a central longitudinal axis of cap 300. In other examples, portions of cap 300 that are adjacent to first terminating end 313 and second terminating end 317 may be approximately perpendicular to the central longitudinal axis of cap 300.

As described in detail below, when cap 300 is coupled to catheter 200 to form at least a portion of an articulating ultrasound imaging device, first terminating end 313 and second terminating end 317 may be positioned proximal to a fixed location at which distal portion 122 of probe 120, including transducer 124, is positioned within or relative to the articulating ultrasound imaging device. The proximal positioning helps to ensure that the fluid expelled via first distal opening 314 and second distal opening 318 at first terminating end 313 and second terminating end 317 of cap 300, respectively, flushes out (e.g., replaces) air such that the fluid at least partially radially surrounds the transducer 124. Such flushing may supplement and enhance effects of the fluid expelled via distal opening 308 of central lumen 306 for improved sound wave transmission.

An interior of cap 300 may include a step 330 (e.g., a ledge or a shelf) or other similar structure. In some embodiments, the step 330 may be configured for receiving and securing a distal component of the articulating ultrasound imaging device to cap 300 and catheter 200, as described in more detail with reference to FIGS. 5A-D below. As shown, step 330 may be an annular step, where an inner diameter of step 330 corresponds to central lumen 306 such that probe 120 may still be enabled to extend through and distally past central lumen 306.

As shown in FIGS. 3B and 3C, cap 300 may also include one or more articulation cavities, including a first articulation cavity 320 and a second articulation cavity 324. First articulation cavity 320 may distally extend a first distance from a first articulation opening 322 located on a proximal-facing surface of proximal end 302 partially through cap 300. Second articulation cavity 324 may distally extend the first distance from a second articulation opening 326 located on a proximal-facing surface of proximal end 302 partially through cap 300.

As shown and described with reference to FIGS. 2A-2B, the distal ends of first articulation wire 252 and second articulation wire 254 may extend distally past first articulation lumen 220 and second articulation lumen 224 via third distal wall opening 222 and fourth distal wall opening 226. First articulation opening 322 and second articulation opening 326 of cap 300 may correspond to (align with) third distal wall opening 222 and fourth distal wall opening 226, respectively. Accordingly, the distal ends of first articulation wire 252 and second articulation wire 254 may be received by first articulation cavity 320 and second articulation cavity 324 via first articulation opening 322 and second articulation opening 326, respectively. Distal ends of first articulation wire 252 and second articulation wire 254 may then be secured within (e.g., to a wall of) first articulation cavity 320 and second articulation cavity 324 using one or more attachment or fixing mechanisms (e.g., adhesives, welding, etc.) to couple first articulation wire 252 and second articulation wire 254 to cap 300.

In some examples and as described in more detail below, an articulating ultrasound imaging device, formed at least partially by catheter 200, cap 300, and articulation mechanism 250, may be inserted into and navigated through a body lumen (e.g., through an airway in a peripheral lung region). The steerability provided by articulation mechanism 250, in addition to a rigidity of at least catheter 200 may facilitate such navigation. Further, when the articulating ultrasound imaging device is positioned near an intended target, steerability provided by articulation mechanism 250 may enable adjustment of a position of probe 120, and specifically transducer 124, relative to the target area to help improve image quality. Specific examples of articulating ultrasound imaging devices formed at least partially by catheter 200, cap 300, and/or articulation mechanism 250, are described in turn below.

FIGS. 4A-4C depict second ultrasound imaging device 400, hereinafter device 400 for brevity. Device 400 may replace (e.g., may be used instead of or interchanged with) device 102 in system 100. For example, device 400 may be electrically and/or mechanically connected to control unit 130, display device 140, and/or fluid source 150, as described in detail above with reference to FIG. 1A. Device 400 may include two separate devices, first device 402 and second device 404. Each of first device 402 and second device 404 may be single use or disposable device such that device 400 as a whole is a single use device. Alternatively, one of first device 402 or second device 404 may be a reusable device. FIG. 4A depicts a perspective view of a distal portion of first device 402. FIG. 4B depicts a perspective view of second device 404 received by and extending distally from distal portion of first device 402. FIG. 4C depicts a cross-sectional view of device 400 taken along line 4C in FIG. 4B.

Referring concurrently to FIGS. 4A-4C, first device 402 may be a multi-lumen component, including catheter 200 coupled to corresponding cap 300. As described in detail above with reference to FIGS. 2A-2B and FIGS. 3A-3C, first flushing lumen 212 of catheter 200 may be in fluid communication with fluid source 150 via first proximal wall opening 213, and may be extended by first flushing lumen 312 of cap 300 to first terminating end 313 that includes first distal opening 314 (e.g., forming the continuous first flushing lumen). Similarly, second flushing lumen 216 of catheter 200 may be in fluid communication with fluid source 150 via second proximal wall opening 217, and may be extended by second flushing lumen 316 of cap 300 to second terminating end 317 that includes second distal opening 318 (e.g., forming the continuous second flushing lumen). Therefore, fluid provided from fluid source 150 may be received by and flow distally through the continuous first and second flushing lumens, and out of first distal opening 314 and second distal opening 318, respectively.

Additionally, first device 402 may be an articulating, multi-lumen component that further includes an articulation mechanism, such as articulation mechanism 250, described above in detail with reference to FIG. 2B, that is integrated with catheter 200 and cap 300. For example, and as shown in FIG. 4C, distal ends of first articulation wire 252 and second articulation wire 254 may be received by and extended through first articulation lumen 220 and second articulation lumen 224, respectively, of catheter 200. Corresponding first articulation cavity 320 and second articulation cavity 324 of cap 300 may then be configured to receive and secure distal ends of first articulation wire 252 and second articulation wire 254 to cap 300. Proximal ends of first articulation wire 252 and second articulation wire 254 may be attached to actuator 258 (FIG. 2B) to enable actuation thereof to move articulation region of first device 402 in at least one directional plane (e.g., up and down, left or right, etc.), as described in more detail below. The articulation region may include at least cap 300. In some examples, the articulation region may also include a distal portion of catheter 200 coupled to cap 300.

Referring now to FIGS. 4B-4C, second device 404 may include a catheter 405 having a single lumen 406 (e.g., a single-lumen catheter) and probe 120 that is received by and positioned at a fixed location within lumen 406 of catheter 405 (e.g., a visualization region or imaging window). In some examples, second device 404 may be a commercially available ultrasound device. Second device 404 may be removably inserted into and extended through first device 402 via central lumen 206 (FIGS. 2A-B) of catheter 200 and central lumen 306 (FIGS. 3A-C) of cap 300. In some examples, second device 404 may be less rigid (e.g., more flexible) than first device 402.

Catheter 405 may also be in fluid communication with fluid source 150. For example, catheter 405 maybe in fluid communication with fluid source 150 via a proximal opening (not shown) of lumen 406. Resultantly, when fluid is provided from fluid source 150, fluid may also be received by and flow distally through lumen 406, and be expelled from catheter 405 via distal opening 408 of lumen 406. The fluid may flow over probe 120, positioned within lumen 406, as the fluid flows toward and out of distal opening 408.

In a configuration of device 400 in which transducer 124 is active, a distal portion of second device 404 may be extended distally past a distalmost end of first device 402. In some examples, an outer surface of catheter 405 and/or an inner surface of lumen 406 may include one or more stopping structures (not shown), such as barbs, stoppers, protrusions, or other similar structures. As second device 404 is inserted into and extended through first device 402, the stopping structures may limit a distance to which second device 404 may be extended distally from first device 402. For example, the stopping structures may be positioned at a location on the outer surface of catheter 405 at a predefined distance proximal from the location of transducer 124 at distal portion 122 of probe 120 within lumen 406. The predefined distance may help to ensure that fluid expelled from the continuous first and second flushing lumens via first distal opening 314 and second distal opening 318 flushes out (e.g., replaces) air such that the fluid at least partially radially surrounds the portion of catheter 405 including transducer 124, which may supplement and enhance the fluid provided via lumen 406 for improved sound wave transmission. Furthermore, catheter 405 may have a narrower outer diameter/width than a diameter/width of a portion of cap 300 defining first distal opening 314 and second distal opening 318, such that fluid from first distal opening 314 and second distal opening 318 may surround catheter 405 and transducer 124.

In some examples, first device 402 may be inserted into and navigated through a body lumen (e.g., through an airway in a peripheral lung region) to an intended target area independently and prior to insertion of second device 404 into first device 402. Alternatively, second device 404 may be inserted into first device 402 prior to navigation through the body lumen. The steerability provided by articulation mechanism 250 included and/or integrated with first device 402, in addition to the rigidity of first device 402 provided by at least catheter 200, may facilitate such navigation. Further, when distal portion of second device 404 is extended through and distally past first device 402, and is positioned near an intended target area for imaging, actuator 258 on handle 256 may be actuated by an operator of device 400. Such actuation may cause movement of distal portion of first device 402 (e.g. up and down or left and right), which causes corresponding movement of second device 404 in order to adjust a position of transducer 124 relative to the target area to help improve image quality.

FIGS. 5A-5D depict third ultrasound imaging device 500, hereinafter device 500 for brevity. Device 500 may replace (e.g., may be used instead of or interchanged with) device 102 in system 100. For example, device 500 may be electrically and/or mechanically connected to control unit 130, display device 140, and/or fluid source 150. Device 500 may be a single use or disposable device. FIG. 5A depicts a perspective view of device 500. FIG. 5B depicts a partially transparent perspective view of device 500. FIG. 5C depicts a cross-sectional view of device 500 taken along line 5C in FIG. 5B. FIG. 5D depicts a front view of a distal end 503 of device 500.

Referring concurrently to FIGS. 5A-5D, device 500 may include a multi-lumen component 502 attached to a proximal end of a single-lumen tube or catheter 504. Multi-lumen component 502 may include catheter 200 coupled to corresponding cap 300. As described in detail above with reference to FIGS. 2A-2B and FIGS. 3A-3C, first flushing lumen 212 of catheter 200 may be in fluid communication with fluid source 150 via first proximal wall opening 213, and may be extended by first flushing lumen 312 of cap 300 to first terminating end 313 that includes first distal opening 314 (e.g., forming the continuous first flushing lumen). Similarly, second flushing lumen 216 of catheter 200 may be in fluid communication with fluid source 150 via second proximal wall opening 217, and may be extended by second flushing lumen 316 of cap 300 to second terminating end 317 that includes second distal opening 318 (e.g., forming the continuous second flushing lumen). Therefore, fluid provided from fluid source 150 may be received by and flow distally through the continuous first and second flushing lumens, and out of first distal opening 314 and second distal opening 318, respectively.

Single-lumen catheter 504 may include a central lumen 506 having a proximal opening 507 (FIG. 5C) and a distal opening 508. A proximal end of single-lumen catheter 504 may be received by and secured to cap 300 using a fixing mechanism (e.g., adhesive, welding, etc.). For example, and as shown in FIG. 5C, a proximal end of single-lumen catheter 504 may be received by and secured to central lumen 306 of cap 300, with a proximalmost end of single-lumen catheter 504 abutting step 330 of cap 300. Proximal opening 507 of central lumen 506 may correspond to (be aligned with) distal opening 308 of central lumen 306 of cap 300. Resultantly, central lumen 506 may extend central lumen 206 of catheter 200 and central lumen 306 of cap 300 to distal end 503 of device. In other words, central lumen 206 may be a first portion, central lumen 306 may be a second portion, and central lumen 506 may be a third portion of a continuous central lumen extending from proximal end of device 500 to distal end 503 of device 500.

The continuous central lumen may be configured to receive probe 120. Additionally, proximal opening 207 of central lumen 206 at the proximal end of device 500 may be in fluid connection with fluid source 150. Resultantly, fluid received from fluid source 150 via proximal opening 207 may flow distally from proximal end of device 500 to distal end 503 of device 500 via the continuous central lumen, and expelled out of distal opening 508. Thus, the fluid may flow over probe 120 received within the continuous central lumen to help improve sound wave transmission.

In some aspects, probe 120 may be positioned at a fixed location within the continuous central lumen such that first terminating end 313 and second terminating end 317 of the continuous first and second flushing lumens are proximal from transducer 124. For example, when probe 120 is positioned at the fixed location, distal portion 122 of probe 120, including transducer 124, may be positioned within central lumen 506 of single-lumen catheter 504 toward distal end 503 of device 500. The proximal positioning may help to ensure that the fluid expelled from the continuous first and second flushing lumens via first distal opening 314 and second distal opening 318 flushes out (e.g., replaces) air such that the fluid at least partially radially surrounds the portion of single-lumen catheter 504 including transducer 124 (e.g., surrounds a visualization region or imaging window). Such proximal flushing of fluid may supplement and enhance the fluid provided via the continuous central lumen and expelled from distal opening 508 for further improved sound wave transmission. Furthermore, single-lumen catheter 504 may have a narrower outer diameter/width than a diameter/width of a portion of cap 300 defining first distal opening 314 and second distal opening 318, such that fluid from first distal opening 314 and second distal opening 318 may surround single-lumen catheter 504 and transducer 124.

Additionally, multi-lumen component 502 may be an articulating, multi-lumen component that further includes an articulation mechanism, such as articulation mechanism 250, described above in detail with reference to FIG. 2B, that is integrated with catheter 200 and cap 300. For example, and as shown in FIG. 5C, distal ends of first articulation wire 252 and second articulation wire 254 may be inserted into and extended through first articulation lumen 220 and second articulation lumen 224, respectively, of catheter 200. Corresponding first articulation cavity 320 and second articulation cavity 324 of cap 300 may then be configured to receive and secure distal ends of first articulation wire 252 and second articulation wire 254 to cap 300. Distal ends of first articulation wire 252 and second articulation wire 254 may be secured to cap 300 using a fixing mechanism (e.g., using an adhesive, welding, etc.). Proximal ends of first articulation wire 252 and second articulation wire 254 may be attached to actuator 258 (FIG. 2B) to enable actuation thereof to steer or move an articulation region at a distal portion of multi-lumen component 502 in at least one directional plane (e.g., up and down, left or right, etc.). The articulation region may include at least cap 300. In some examples, the articulation region may also include a distal portion of catheter 200 coupled to cap 300.

In some examples, device 500 may be inserted into and navigated through a body lumen (e.g., through an airway in a peripheral lung region) to an intended target area. The steerability provided by articulation mechanism 250 included and/or integrated with device 500, in addition to a rigidity of device 500 provided by at least catheter 200, may facilitate such navigation. Further, when distal end 503 of device 500 is positioned near the intended target area for imaging, actuator 258 on handle 256 may be actuated by an operator of device 400. Such actuation may cause movement of the articulation region at the distal portion of multi-lumen component 502 (e.g. up and down or left and right), which causes corresponding movement of single-lumen catheter 504 in order to adjust a position of transducer 124 relative to the target area to help improve image quality.

FIGS. 6A-6D depict a fourth ultrasound imaging device 600, hereinafter device 600 for brevity. Device 600 may replace (e.g., may be used instead of interchanged with) device 102 in system 100. For example, device 600 may be electrically and/or mechanically connected to control unit 130, display device 140, and/or fluid source 150. Device 600 may be a single use or disposable device. FIG. 6A depicts a perspective view of a distal portion of device 600. FIG. 6B depicts a partially transparent perspective view of the distal portion of device 600. FIG. 6C depicts a cross-sectional view of device 600 taken along line 6C in FIG. 6B. FIG. 6D depicts a front view of a distal end 603 of device 600.

Referring concurrently to FIGS. 6A-6D, device 600 may include a multi-lumen catheter, such as catheter 200, attached to a proximal end of a distal component 601. In some examples, and as described in more detail below, distal component 601 may be an overmolded component. Distal component 601 may include a proximal portion 602 coupled to catheter 200, and a distal portion 604 extending from proximal portion 602 to distal end 603 of device 600. Distal component 601 may be similar to cap 300 and single-lumen catheter 504, combined into one single, integral piece. Distal component 601 may have any of the features of cap 300 and/or single-lumen catheter 504. Distal component 601 may also include a central lumen 606 extending through proximal portion 602 and distal portion 604.

Central lumen 606 may include a proximal opening 607 and a distal opening 608. Proximal opening 607 of lumen 606 of distal component 601 may correspond to (be aligned with) distal opening 208 of central lumen 206 of catheter 200. Resultantly, central lumen 606 may extend central lumen 206 to distal end 603 of device 600. In other words, central lumen 206 of catheter 200 and central lumen 606 of distal component 601 may form one continuous central lumen from proximal end (not shown) of device 600 to distal end 603 of device 600. The continuous central lumen may be configured to receive probe 120. Additionally, proximal opening 207 of central lumen 206 (FIG. 2B) may be in fluid connection with fluid source 150. Resultantly, fluid received from fluid source 150 via proximal opening 207 may flow distally from proximal end of device 600 to distal end 603 of device 600 via the continuous central lumen, and expelled out of distal opening 608. Thus, the fluid may flow over probe 120 received within the continuous central lumen to help improve sound wave transmission.

Proximal portion 602 of distal component 601 may be in a form of a cap and include features similar to cap 300. For example, proximal portion 602 may include a first flushing lumen 612 (having any of the properties of first flushing lumen 312) that extends from a proximal end of proximal portion 602 to a first terminating end 613 proximal of distal portion 604 of distal component 601. First terminating end 613 may include a first distal opening 614 that is distal-facing. First distal opening 614 may be proximal of a fixed location at which the distal portion 122 of probe 120 is positioned within lumen 606. For example, first distal opening 614 may be a first predefined distance proximal to transducer 124.

Proximal portion 602 may also include a second flushing lumen 616 (having any of the properties of second flushing lumen 316) that extends from a proximal end of proximal portion 602 to a second terminating end 617 proximal of distal portion 604 of distal component 601. Second terminating end 617 may include a second distal opening 618 that is distal-facing. Second distal opening 618 may be proximal of a fixed location at which the distal portion 122 of probe 120 is positioned within lumen 606. For example, second distal opening 618 may be a second predefined distance proximal to transducer 124. In some examples, the first distal opening 614 and second distal opening 618 may be the same predefined distance proximal to transducer 124 (e.g., the first and second predefined distances may be substantially equal). In other words, first distal opening 614 and second distal opening 618 may be at a same axial position along device 102.

First flushing lumen 212 of catheter 200 may be in fluid communication with fluid source 150 via first proximal wall opening 213 (FIG. 2B), and may be extended by first flushing lumen 612 to first terminating end 613 of proximal portion 602 of distal component 601. For example, a proximal opening of first flushing lumen 612 of proximal portion 602 may correspond to (be aligned with) a distal opening of first flushing lumen 212 of catheter 200 to form a continuous first flushing lumen from a proximal end of device 600 to first terminating end 613 of device 600. Similarly, second flushing lumen 216 of catheter 200 may be in fluid communication with fluid source 150 via second proximal wall opening 217 (FIG. 2B), and may be extended by second flushing lumen 616 to second terminating end 617 of proximal portion 602 of distal component 601. For example, a proximal opening of second flushing lumen 616 of proximal portion 602 may correspond to (be aligned with) a distal opening of second flushing lumen 216 of catheter 200 to form a continuous second flushing lumen from a proximal end of device 600 to second terminating end 617 of device 600. Therefore, fluid provided from fluid source 150 may be received by and flow distally through the continuous first flushing lumen and the continuous second flushing lumen, and out of first distal opening 614 and second distal opening 618, respectively.

In some aspects, first terminating end 613 and second terminating end 617 of proximal portion 602 of distal component 601 may be positioned proximal of a fixed location at which the distal portion 122 of probe 120 is positioned within the continuous central lumen. For example, distal portion 122 of probe 120, including transducer 124, may be positioned within distal portion 604 of distal component 601 toward distal end 603 of device 600. The proximal positioning of first terminating end 613 and second terminating end 617 may help to ensure that the fluid expelled from the continuous first and second flushing lumens via first distal opening 614 and second distal opening 618, respectively, flushes out (e.g., replaces) air such that the fluid at least partially radially surrounds distal portion 604 of distal component 601 including transducer 124 (e.g., surrounds a visualization region or imaging window). Such proximal flushing of fluid may supplement and enhance the fluid provided via the continuous central lumen and expelled from distal opening 608 for further improved sound wave transmission. Similar to single-lumen catheter 504, distal portion 604 may have a smaller outer diameter/width than a portion of proximal portion 602 that defines first distal opening 614 and second distal opening 618, such that fluid from first distal opening 614 and second distal opening 618 may surround distal portion 604 and transducer 124.

Additionally, device 600 may include and/or may be integrated with an articulation mechanism, such as articulation mechanism 250, described above with reference to FIG. 2B. In such examples, proximal portion 602 of distal component 601 may also include a first articulation cavity 620 and a second articulation cavity 624. For example, and as shown in FIG. 6C, distal ends of first articulation wire 252 and second articulation wire 254 may be inserted into and extended distally through first articulation lumen 220 and second articulation lumen 224, respectively, of catheter 200. First articulation cavity 620 and second articulation cavity 624 of proximal portion 602 of distal component 601, shown in FIGS. 6B and 6C, that correspond to (align with) first articulation lumen 220 and second articulation lumen 224, respectively, may then be configured to receive distal ends of first articulation wire 252 and second articulation wire 254. Distal ends of first articulation wire 252 and second articulation wire 254 may be secured to a distal wall of first articulation cavity 620 and second articulation cavity 624. Proximal ends of first articulation wire 252 and second articulation wire 254 may be attached to actuator 258 (FIG. 2B) to enable actuation thereof to steer or move an articulation region at a distal portion of device 600 in at least one directional plane (e.g., up and down, left or right, etc.). The articulation region may include at proximal portion 602 of distal component 601. In some examples, the articulation region may also include a distal portion of catheter 200 coupled to proximal portion 602 of distal component 601.

In some examples, device 600 may be inserted into and navigated through a body lumen (e.g., through an airway in a peripheral lung region) to an intended target area. The steerability provided by articulation mechanism 250 included and/or integrated with device 600, in addition to a rigidity of device 600 provided by at least catheter 200, may facilitate such navigation. Further, when distal end 603 of device 600 is positioned near the intended target area for imaging, actuator 258 on handle 256 may be actuated by an operator of device 400. Such actuation may cause movement of the articulation region at the distal portion of device 600 (e.g. up and down or left and right), which causes corresponding movement of distal portion 604 of distal component 601 in order to adjust a position of transducer 124 relative to the target area to help improve image quality.

In some aspects, distal component 601 may be formed via injection molding. In some examples, proximal portion 602 may be in the form of a cap, similar to cap 300, when positioned with catheter 200 into the mold. In such examples, catheter 200 and proximal portion 602 having first articulation wire 252 and second articulation wire 254 integrated therewith may be positioned within a mold and injected with a material. The mold may be configured to enable the material to melt, for example, to form distal portion 604 (and in some examples proximal portion 602), and join proximal portion 602 with distal portion 604 to form distal component 601. Additionally, the mold may further be configured to join at least a portion of distal component 601 to catheter 200 to, for example, secure or otherwise couple distal component 601 to catheter 200 to form device 600.

In other examples, both proximal portion 602 and distal portion 604 may be formed by the mold. In such examples, catheter 200 having first articulation wire 252 and second articulation wire 254 integrated therewith may be positioned within a mold and injected with a material. The mold may be configured to enable the material to melt, for example, to form proximal portion 602 and distal portion 604 as a single distal component 601. Additionally, the mold may further be configured to join at least a portion of distal component 601 to catheter 200 to, for example, secure or otherwise couple distal component 601 to catheter 200 to form device 600.

In either of the above examples, distal component 601 may be formed and coupled to catheter 200 prior to probe 102 being received by and positioned at the fixed location within the continuous central lumen formed by central lumen 206 of catheter 200 and central lumen 606 of distal component 601.

In other aspects, other known or future manufacturing processes, such as heat shrinking, reflow, etc., may be utilized to form distal component 601 and/or device 600. In examples, where injection molding or other similar manufacturing processes are utilized to form device 600, catheter 200 and distal component 601, including both proximal portion 602 and distal portion 604, may be formed from thermoplastic materials, such as Pebax and/or Nylon.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
a central lumen extending from a proximal end to a distal end of the medical device, and configured to receive a probe, wherein a distal portion of the probe includes a transducer configured to perform ultrasound imaging, and wherein the transducer is positioned at a fixed location relative to the medical device in a configuration in which the probe is received by the central lumen; and
a flushing lumen extending from the proximal end of the medical device to a distalmost opening of the flushing lumen, wherein the flushing lumen is disposed radially outward of the central lumen, wherein the flushing lumen is in fluid communication with a fluid source at the proximal end of the medical device, and wherein the distalmost opening is proximal of the fixed location such that a flow of fluid received from the fluid source and expelled by the flushing lumen via the distalmost opening at least partially radially surrounds the transducer.

2. The medical device of claim 1, wherein the flushing lumen is a first flushing lumen extending from the proximal end of the medical device to a first distalmost opening proximal from the fixed location, and the medical device further comprises:
a second flushing lumen extending from the proximal end of the medical device to a second distalmost opening of the second flushing lumen, and disposed radially outward of the central lumen, wherein the second flushing lumen is in fluid communication with the fluid source at the proximal end of the medical device, and the second distalmost opening is proximal from the fixed location.

3. The medical device of claim 1, wherein the central lumen is in fluid communication with the fluid source at the proximal end of the medical device, and wherein the central lumen is further configured to provide a flow of fluid received from the fluid source to the distal end of the medical device through the central lumen.

4. The medical device of claim 1, wherein the medical device comprises:
an outer sheath; and
an inner sheath that is positioned within and extends distally from the outer sheath, wherein the central lumen is a lumen of the inner sheath, and wherein the flushing lumen is between the inner sheath and the outer sheath.

5. The medical device of claim 1, wherein the medical device comprises:
a multi-lumen catheter including a first portion of the central lumen and a first portion of the flushing lumen; and
a cap coupled to the multi-lumen catheter, the cap including a second portion of the central lumen and a second portion of the flushing lumen to extend the flushing lumen to the distalmost opening.

6. The medical device of claim 5, wherein the medical device further comprises:
an articulation wire coupled to the cap, wherein the articulation wire is configured to articulate a distal portion of the medical device.

7. The medical device of claim 6, wherein the multi-lumen catheter further includes an articulation lumen, wherein the articulation wire extends through the articulation lumen, and wherein a distal end of the articulation wire extends distal to a distalmost end of the articulation lumen; and
wherein the cap further includes an articulation cavity aligned with the articulation lumen, wherein the articulation cavity receives the distal end of the articulation wire, and wherein the distal end of the articulation wire is secured to the cap.

8. The medical device of claim 7, wherein at least a distal portion of the multi-lumen catheter and the cap form an articulation region, and wherein the distal portion of the multi-lumen catheter comprises a first material that is more flexible than a second material of which a proximal portion of the multi-lumen catheter is comprised.

9. The medical device of claim 5, wherein the multi-lumen catheter and the cap coupled to the multi-lumen catheter is a first device, and wherein the medical device further comprises a second device including:
a single-lumen catheter; and
the probe, wherein the probe is received within a lumen of the single-lumen catheter, wherein the single-lumen catheter is configured to be received by and extended through the central lumen of the first device, such that a distal portion of the single-lumen catheter including the transducer extends distally past the first device.

10. The medical device of claim 9, wherein, in a configuration in which the transducer is active, the distal portion of the single-lumen catheter is positioned such that the distalmost opening of the flushing lumen of the first device is positioned at a predefined distance proximal to the transducer.

11. The medical device of claim 5, wherein the multi-lumen catheter is coupled to a proximal end of the cap, and wherein the medical device further comprises:
a single-lumen catheter coupled to and extending distally from a distal end of the cap to the distal end of the medical device, the single-lumen catheter including a third portion of the central lumen, wherein, when the probe is received by the central lumen, the fixed location at which the transducer is positioned is within the single-lumen catheter.

12. The medical device of claim 1, wherein the medical device comprises:
a multi-lumen catheter including a first portion of the central lumen and a first portion of the flushing lumen; and
an overmolded component attached distally to the multi-lumen catheter.

13. The medical device of claim 12, wherein the overmolded component comprises:
a proximal portion including a second portion of the central lumen and a second portion of the flushing lumen to extend the flushing lumen to the distalmost opening; and
a distal portion including a third portion of the central lumen.

14. The medical device of claim 13, wherein, when the probe is received by the central lumen, the fixed location at which the transducer is positioned is within the distal portion of the overmolded component.

15. The medical device of claim 13, further comprising:
an articulation wire integrated with the multi-lumen catheter and the proximal portion of the overmolded component, wherein the articulation wire is configured to articulate a distal portion of the medical device.

16. A medical device comprising:
a proximal multi-lumen catheter including:
a central lumen; and
a flushing lumen disposed radially outward of the central lumen, and in fluid communication with a fluid source at a proximal end of the medical device;
a distal single-lumen catheter extending the central lumen from the proximal end to a distal end of the medical device;
a cap configured to couple the proximal multi-lumen catheter to the distal single-lumen catheter, the cap extending the flushing lumen to a distalmost opening of the flushing lumen; and
a probe including a transducer at a distal portion of the probe, wherein the probe is received by and extends through the central lumen toward the distal end of the medical device, wherein the transducer is positioned at a fixed location within the distal single-lumen catheter, and wherein the distalmost opening of the flushing lumen is proximal of the fixed location such that a flow of fluid received from the fluid source and expelled by the flushing lumen via the distalmost opening at least partially radially surrounds the transducer.

17. The medical device of claim 16, wherein the cap includes a step configured to receive and secure the distal single-lumen catheter to the cap.

18. The medical device of claim 16, wherein the cap and the distal single-lumen catheter comprise a proximal portion and a distal portion, respectively, of an overmolded component of the medical device.

19. The medical device of claim 16, further comprising:
an articulation wire integrated with the proximal multi-lumen catheter and the cap.

20. A medical device, comprising:
a multi-lumen catheter, including:
a central lumen; and
a flushing lumen disposed radially outward of the central lumen, and in fluid communication with a fluid source at a proximal end of the medical device; and
a cap coupled to the multi-lumen catheter, the cap extending the central lumen and the flushing lumen to a distal end of the medical device;
wherein at least a distal portion of a single-lumen catheter, separate from the medical device and having a probe including a transducer positioned at a fixed location in the distal portion of the single-lumen catheter, is configured to be removably extended through the central lumen and past the distal end of the medical device, such that a flow of fluid received from the fluid source and expelled by the flushing lumen at the distal end of the medical device at least partially radially surrounds the distal portion of the single-lumen catheter.
